(19) United States
(12) Patent Application Publication
(10) Pub. No.: US 2014/0296794 A1
(43) Pub. Date: Oct. 2, 2014

(54) NEEDLELESS POSITIVE-PRESSURE CONNECTOR

(71) Applicant: Sunwell Global Limited, Tortola (VG)

(72) Inventor: ZHI-YUN LI, JIANGSU PROVINCE (CN)

(73) Assignee: Sunwell Global Limited, Tortola (VG)

(21) Appl. No.: 14/231,926

(22) Filed: Apr. 1, 2014

(30) Foreign Application Priority Data
Apr. 1, 2013 (CN) 201320157672.4

(51) Int. Cl. A61M 39/22

(52) U.S. Cl. CPC A61M 39/22
USPC 604/256

(57) ABSTRACT

The present invention is related to a needleless positive-pressure connector, mainly comprising a casing pipe, a connecting pipe, an elastic unit, and a piston. In this connection, the casing pipe and the connecting pipe are interconnected, while the piston and the elastic unit are located within the interior of the casing pipe and the connecting pipe, as well as the piston may be displaced within the casing pipe and the connecting pipe. The piston may be pushed outwardly by the elastic unit to close the needleless positive-pressure connector, when a delivery pipe is not connected yet Moreover, the piston may be pressed by the delivery pipe, such that the piston is pressed inwardly to open the needleless positive-pressure connector, when the delivery pipe is connected.

### TECHNICAL FIELD

The present invention is related to a needleless positive-pressure connector, capable of enhancing convenience for medical personnel to perform transfusion or injection effectively, and reducing tube-blocking rate of the needleless positive-pressure connector.

### DESCRIPTION OF THE PRIOR ART

During the treatment of venous transfusion, it is frequently necessary for chronic transfusion or injection to the patient. Generally, a vein detained needle or detained trocar is selected to insert into the patient's body. The vein detained needle or detained trocar is connected with a three-way trocar, which is usually equipped with Luer taper or heparin cap, so as to facilitate transfusion of several kinds of liquids or medicaments into the patient's body by medical personnel.

There are still certain drawbacks when the above medical instruments are used clinically. Taking Luer taper on the three-way trocar as an example, local sterilization is required for Luer taper before transfusion is performed through Luer taper, and additional puncture with a needle is further required during operation. In this way, working load and operational risk may be added to medical personnel.

As for heparin cap, injecting heparin or physiological saline to seal tube is required for the heparin cap after medical liquid is injected by medical personnel, so as to prevent thrombus generated due to blood coagulation. Thus, load is similarly added to medical personnel clinically Furthermore, during the process of transfusion performed by means of heparin cap, a little air may be caused probably to enter into the blood circulatory system, thus increasing risk in operation. Moreover, backflow of blood in human body into the detained trocar may be resulted from negative pressure produced when the needle is withdrawn, possibly further generating serious potential safety hazard to the patient

### SUMMARY OF THE INVENTION

It is one object of the present invention to provide a needleless positive-pressure connector, in which a casing pipe and a connecting pipe are interconnected, while a piston and an elastic unit are located within the interior of the casing pipe and the connecting pipe. When the needleless positive-pressure connector is not connected to a delivery pipe, the piston may be pushed outwardly by the elastic unit to close the casing pipe, thus separating the needleless positive-pressure connector from the outside.

It is one object of the present invention to provide a needleless positive-pressure connector, in which only insertion of a needleless delivery pipe, such as transfusion set or syringe, into an inlet is necessary when the needleless positive-pressure connector is used for transfusion. Thus, a piston is pressed to be withdrawn inwardly for opening the needleless positive-pressure connector, and liquid within the delivery pipe is then delivered into the patient's body through the needleless positive-pressure connector.

It is one object of the present invention to provide a needleless positive-pressure connector, in which an outlet of the needleless positive-pressure connector is connected to a vein detained needle, detained trocar, or three-way trocar. It is only necessary for the completion of transfusion to insert a delivery pipe into an inlet of the needleless positive-pressure connector in operation. In addition, the inlet may be closed by the piston, and liquid within a casing pipe may be forced into the patient's body, when the delivery pipe is withdrawn out of the inlet of the needleless positive-pressure connector. Thus, thrombus generated due to backflow of blood may be avoided, so as to reduce tube-blocking rate of the needleless positive-pressure connector.

It is one object of the present invention to provide a needleless positive-pressure connector, used for connecting to a trocar, detained needle, or detained trocar, and eliminating repeated process of sealing tube and washing tube. Thus, working load of medical personnel for nursing may be reduced effectively, so as to enhance work efficiency.

To achieve these and other objects of the present invention, the present invention provides a needleless positive-pressure connector, comprising: a connecting pipe comprising an outlet and an extension portion, wherein the extension portion is provided therein with a first accommodating room; a casing pipe comprising an inlet and a second accommodating room, wherein the extension portion of the connecting pipe is located within the second accommodating room when the casing pipe and the connecting pipe are coupled; a piston connected to the connecting pipe, a part of the piston being located within the first accommodating room, wherein the piston is used for displacing within the first accommodating room and the second accommodating room, as well as opening or closing the inlet, and an elastic unit, located between the connecting pipe and the piston.

In one embodiment of the needleless positive-pressure connector, the connecting pipe comprises a projection portion located within the first accommodating room, while the piston comprises a depressed portion, as well as one end of the elastic unit is located at the projection portion, while the other end of the elastic unit is located within the depressed portion.

In one embodiment of the needleless positive-pressure connector, the piston is used for separating the first accommodating room from the second accommodating room.

In one embodiment of the needleless positive-pressure connector, the piston is used for separating the first accommodating room from the outlet.

In one embodiment of the needleless positive-pressure connector, further comprises at least one connecting hole connecting the second accommodating room to the outlet.

In one embodiment of the needleless positive-pressure connector, further comprises a gap located between the extension portion of the connecting pipe and the casing pipe.

In one embodiment of the needleless positive-pressure connector, further comprises a first sealing unit and a second sealing unit located at two ends of the piston, respectively, the first sealing unit being located within the first accommodating room of the connecting pipe, the second sealing unit facing toward the inlet.

In one embodiment of the needleless positive-pressure connector, the second sealing unit is adhered to an inner wall of the casing pipe, and the second accommodating room is separated from the outside to close the inlet.

In one embodiment of the needleless positive-pressure connector, the connecting pipe further comprises at least one vent connected with the first accommodating room.

In one embodiment of the needleless positive-pressure connector, further comprises at least one notch located at a front end of the piston.

### DETAILED DESCRIPTION OF THE PREFERRED EMBODIMENTS

Referring to FIGS. 1 and 2, there are shown a perspective exploded diagram and a cross-sectional exploded diagram, respectively, of a needleless positive-pressure connector according to one embodiment of the present invention. As illustrated in figures, a needleless positive-pressure connector 10 described in the present invention mainly comprises a connecting pipe 11, a casing pipe 13, an elastic unit 15, and a piston 17. In this connection, the connecting pipe 11 is connected to the casing pipe 13, while the elastic unit 15 and the piston 17 are located within the connecting pipe 11 and/or the casing pipe 13. Moreover, the piston 17 is allowed to perform displacement within the connecting pipe 11 and the casing pipe 13, as well as open or close the needleless positive-pressure connector 10.

The connecting pipe 11 comprises an outlet 111 and an extension portion 113, wherein the extension portion 113 comprises a first accommodating room 12 therein. For instance, the extension portion 113 may be presented as a hollow cylinder The first accommodating room 12 may be used for accommodating the elastic unit 15 and a part of the piston 17. In this case, the piston 17 is allowed to perform displacement relative to the connecting pipe 11 within the first accommodating room 12.

The casing pipe 13 is presented as a hollow pipe, and comprises an inlet 131 and a second accommodating room 14. Inside the second accommodating room 14, a part of the connecting pipe 11 is located, such that the coupling between the connecting pipe 11 and the casing pipe 13 is achieved. When the connecting pipe 11 and the casing pipe 13 are interconnected, the extension portion 113 of the connecting pipe 11 may be located within the second accommodating room 14, while a part of the piston 17 and/or a part of the elastic unit 15 within the extension portion 113 may be located within the second accommodating room 14.

One end of the casing pipe 13, which is not connected to the connecting pipe 11, may be defined as the inlet 131, while one end of the connecting pipe 11, which is not connected to the casing pipe 13, may be defined as the outlet 111. In the practical application of the needleless positive-pressure connector 10, fluid is allowed to flow in via the inlet 131, and flow out via the outlet 111.

The piston 17 is connected to the connecting pipe 11, and a part of the piston 17 may be located within the first accommodating room 12. In the practical application, the piston 17 is allowed to displace within the first accommodating room 12 and the second accommodating room 14, as well as open or close the inlet 131 of the needleless positive-pressure connector 10.

In one embodiment of the present invention, the piston 17 is provided at two ends thereof with a first sealing unit 191 and a second sealing unit 193, respectively. In this connection, the first sealing unit 191 is located within the first accommodating room 12 of the connecting pipe 11, while the second sealing unit 193 is located so as to face toward the inlet 131. In one preferred embodiment of the present invention, referring to FIG. 3 cooperatively, the piston 17 comprises a rear end 171 and a front end 173. Moreover, the rear end 171 and the front end 173 are provided with the first sealing unit 191 and the second sealing unit 193, respectively. For instance, the piston 17 is provided at the rear end 171 thereof with a first groove 172, while at the front end 173 thereof with a second groove 174. In this connection, the first sealing unit 191 is surrounded within the first groove 172, while the second sealing unit 193 is surrounded within the second groove 174.

The rear end 171 of the piston 17 and/or the first sealing unit 191 may be provided within the first accommodating room 12, and displaced within the first accommodating room 12. In this connection, the rear end 171 of the piston 17 and/or the first sealing unit 191 may be adhered to an inner wall 114 of the connecting pipe 11, such that a part of or the whole of first accommodating room 12 may be separated. The front end 173 of the piston 17 and/or the second sealing unit 193 may be then located within the second accommodating room 14. In this connection, the second sealing unit 193 may be adhered to a part of an inner wall 133 of the casing pipe 13, such that a part of or the whole of second accommodating room 14 may be separated, and the inlet 131 is closed.

The piston 17 is connected to the elastic unit 15. In this connection, the elastic unit 15 is located between the piston 17 and the connecting pipe 11, as well as allowed for pushing the piston 17 away from the connecting pipe 11 and/or the first accommodating room 12. When the external force is acted on the piston 17, the elastic unit 15 is compressed, such that the piston 17 is displaced toward the connecting pipe 11 and/or the first accommodating room 12. The detailed operation will be described in the following embodiment.

In one preferred embodiment of the present invention, the elastic unit 15 may be a spring. The piston 17 comprises a depressed portion 175, while the connecting pipe 11 comprises a projection portion 115, the projection portion 115 being located within the first accommodating room 12. During installation, the elastic unit 15 is surroundingly provided at one end thereof onto the projection portion 115, while the elastic unit 15 is located at the other end thereof within the depressed portion 175, so as to achieve interconnection among the connecting pipe 11, the elastic unit 15 and the piston 17, and thus avoid release occurring during operation.

Referring to FIGS. 4 and 5, there are shown cross-sectional diagrams of the needleless positive-pressure connector in the closed state and in the open state, respectively, of the present invention. Referring to FIGS. 1 and 2 cooperatively, the needleless positive-pressure connector 10 described in the present invention mainly comprises the connecting pipe 11, the casing pipe 13, the elastic unit 15, and the piston 17. In this connection, a part of the piston 17 and a part of the elastic unit 15 are located within the first accommodating room 12 of the connecting pipe 11, while a part of the connecting pipe 11 and a part of the piston 17 are located within the second accommodating room 14 of the casing pipe 13.

The needleless positive-pressure connector 10 described in the present invention comprises the inlet 131 and the outlet 111, the outlet 111 being allowed to connect to a trocar 18, such as a detained needle, a locking inner connector, or a detained trocar. In operation, the inlet 131 may be inserted into a delivery pipe 16, such as a transfusion tube or a syringe, so as to deliver liquid within the delivery pipe 16 to the needleless positive-pressure connector 10 from the inlet 131, and then to the trocar 18 and into the patient's body from the outlet 111. In operation, the piston 17 may be displaced relative to the connecting pipe 11 and the casing pipe 13, and allowed to open or close the inlet 131 of the needleless positive-pressure connector 10.

In one embodiment of the present invention, the second accommodating room 14 comprises a first area 141, a second area 143, and a third area 145. In this case, the opening between the first area 141 and the outside is the inlet 131, and the first area 141 is connected to the third area 145 via the second area 143. The cross-sectional area A1 of the first area 141 is smaller than the cross-sectional area A2 of the second area 143, while the cross-sectional area A2 of the second area 143 is smaller than the cross-sectional area A3 of the third area 145.

When the connecting pipe 11 is connected to the casing pipe 13, the extension portion 113 of the connecting pipe 11 is located within the third area 145. In this connection, the volume and/or cross-sectional area of the extension portion 113 may be smaller than the volume and/or cross-sectional area of the third area 145, such that, between the coupled extension portion 113 and the inner wall 133 of the casing pipe 13 within the third area 145, there is existed a gap 1451, via which fluid is delivered.

The piston 17 may be used for separating the first accommodating room 12 from the second accommodating room 14, as well as used for separating the first accommodating mom 12 from the outlet 111. In one embodiment of the present invention, the piston 17 comprises a rear end 171 and a front end 173, in which the cross-sectional area of the rear end 171 is larger than that of the front end 173. The rear end 171 of the piston 17 is located within the first accommodating room 12 of the connecting pipe 11, while the front end 173 of the piston 17 is located within the second accommodating room 14 of the casing pipe 13 and/or the first accommodating room 12 of the connecting pipe 11. For instance, the front end 173 of the piston 17 may be located within the first area 141 and/or the second area 143 of the second accommodating room 14, as well as displaced between the first area 141 and the second area 143.

In one embodiment of the present invention, the cross-sectional area of the front end 173 is slightly smaller than or equivalent to the cross-sectional area A1 of the first area 141. For instance, between the front end 173 of the piston 17 and the inner wall 133 of the casing pipe 13, there is existed a gap, which is sealed by the second sealing unit 193. When the front end 173 is located within the first area 141, the second sealing unit 193 may be adhered to the inner wall 133 of the casing pipe 13 located within the first area 141, so as to close the inlet 131, and then separate the second accommodating room 14 from the outside.

The cross-sectional area of the rear end 171 of the piston 17 is larger than the cross-sectional area Al of the first area 141, and slightly smaller than or equivalent to the cross-sectional area A2 of the second area 143. When the piston 17 is acted by the elastic unit 15 to displace toward the inlet 131, it may be clamped between the first area 141 and the second area 143, in such a way that the piston 17 is never to be separated from the connecting pipe 11 and/or the casing pipe 13, as illustrated in FIG. 4.

In addition, the cross-sectional area of the front end 173 is smaller than the cross-sectional area A2 of the second area 143. When the front end 173 is displaced to the second area 143 (departed from the first area 141), a gap 1431 may be generated between the front end 173 and the inner wall 133 of the casing pipe 13 located within the second area 143, as illustrated in FIG. 5.

To this end, in the case that the needleless positive-pressure connector 10 described in the present invention is not in operation, such as the piston 17 being not acted by the external force, for example, the piston 17 may be displaced toward the inlet 131 due to the action of the elastic unit 15, and clamped between the first area 141 and the second area 143. At this moment, the second sealing unit 193 may be adhered to the inner wall 133 of the casing pipe 13 located within the first area 141, and allowed to separate the second accommodating room 14 from the outside. Then, the needleless positive-pressure connector 10 is presented in the closed state.

When the piston 17 of the needleless positive-pressure connector 10 is acted by the external force, the piston 17 is withdrawn inwardly. For instance, when the pressure is exerted on the piston 17 by the delivery pipe 16 through the inlet 131, the piston 17 is allowed to resist the elastic force of the elastic unit 15, and displaced toward the outlet 111 and/or the connecting pipe 11, as illustrated in FIG. 5. When the second sealing unit 193 is not adhered to the inner wall 133 of the casing pipe 13 anymore, such as the piston 17 and/or the second sealing unit 193 being withdrawn from the first area 141, the second accommodating room 14 may be contacted with the outside, and fluid within the delivery pipe 16 is then allowed to enter into the needleless positive-pressure connector 10, such as into the accommodating room 14 of the casing pipe 13.

The connecting pipe 11 comprises at least one connecting hole 117, used for connecting the second accommodating room 14 to the outlet 111, such that liquid introduced from the delivery pipe 16 is allowed to enter into the gap 1431 between the piston 17 and the easing pipe 13, and then flow to the connecting hole 117 of the connecting pipe 11 through the gap 1451, followed by flowing out of the outlet 111. In addition, the liquid, entering into the needleless positive-pressure connector 10 from the delivery pipe 16, is never allowed to flow into the depressed portion 175, due to the fact that the piston 17 and/or the first sealing unit 191 is still adhered to the inner wall 114 of the connecting pipe 11. Thereby, the contact between the elastic unit 15 and the liquid is avoided. In addition, the connecting pipe 11 may also comprise at least one vent 119, used for connecting the first accommodating room 12 to the outside. When the piston 17 is withdrawn inwardly under external force, the volume of the first accommodating room 12 may be compressed, so as to discharge gas within the first accommodating room 12 through the vent 119.

During the process of departure of delivery pipe 16 out of the inlet 131, the piston 17 may be displaced toward the inlet 131 slowly under the action of the elastic unit 15. After the piston 17 and/or the second sealing unit 193 is allowed to enter into the first area 141, the second sealing unit 193 may be adhered to the inner wall 133 of the casing pipe 13, and allowed to separate the needleless positive-pressure connector 10 from the outside.

In addition, the piston 17 and/or the second sealing unit 193 may be displaced toward the inlet 13 1 along with the delivery pipe 16 subsequent to entering into the first area 141. During the process of displacement, the piston 17, originally located within the first accommodating room 12, may be departed from the first accommodating room 12, to compress liquid within the second accommodating room 14, gap 1431 and/or gap 1451, in such a way that the liquid is delivered to the trocar 18 and into the patient's body from the outlet 111 subsequently. Thereby, blood of patient is prevented from being flowed back to the needleless positive-pressure connector 10 effectively, and probability of thrombus or blocking of needleless positive-pressure connector 10 may be reduced.

In one embodiment of the present invention, the front end 173 of the piston 17 may be provided at least one notch 173 1, referring to FIG. 3 cooperatively. Then, liquid delivered through the delivery pipe 16 may be allowed to flow into the gap 1431/1451, the connecting hole 117 and the outlet 111 through the notch 173 1. In addition, the notch 173l may be projected beyond the inlet 131, as illustrated in FIG. 4, when the piston 17 is displaced toward the inlet 131 under the action of the elastic unit 15.

It is to be understood the invention is not limited to particular systems or methods described which may, of course, vary. It is also to be understood that the terminology used herein is for the purpose of describing particular embodiments only, and is not intended to be limiting. As used in this specification, the singular forms “a”, “an” and “the” include plural referents unless the content clearly indicates otherwise. Thus, for example, reference to “a device” includes a combination of two or more devices and reference to “a material” includes mixtures of materials.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

### BRIEF DESCRIPTION OF DRAWINGS

FIG. 1 is a perspective exploded diagram of a needleless positive-pressure connector according to one embodiment of the present invention;

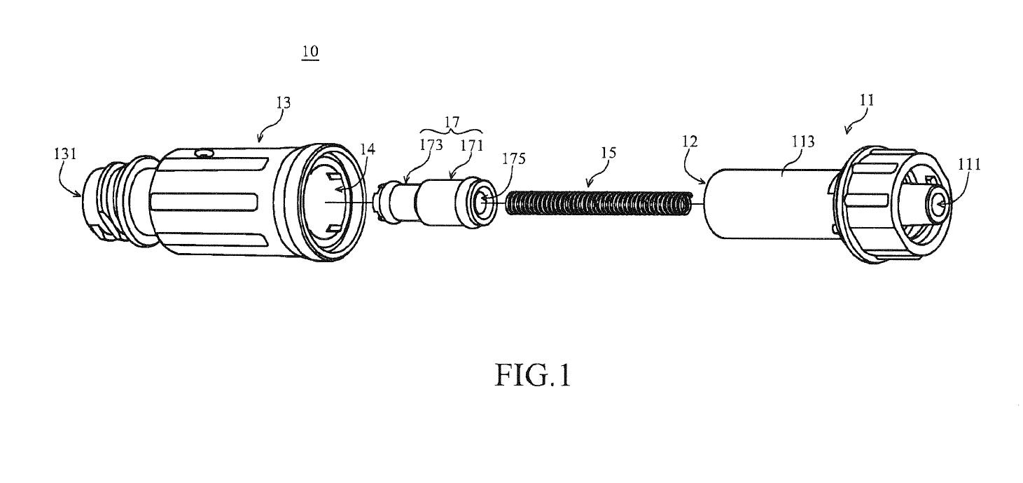

FIG. 2 is a cross-sectional exploded diagram of the needleless positive-pressure connector according to one embodiment of the present invention;

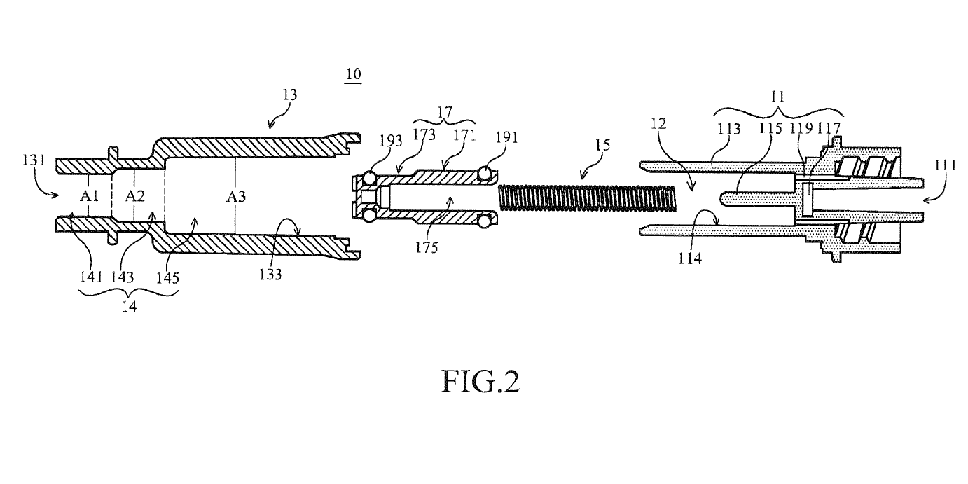

FIG. 3 is a perspective exploded diagram of a part of components of the needleless positive-pressure connector according to one embodiment of the present invention;

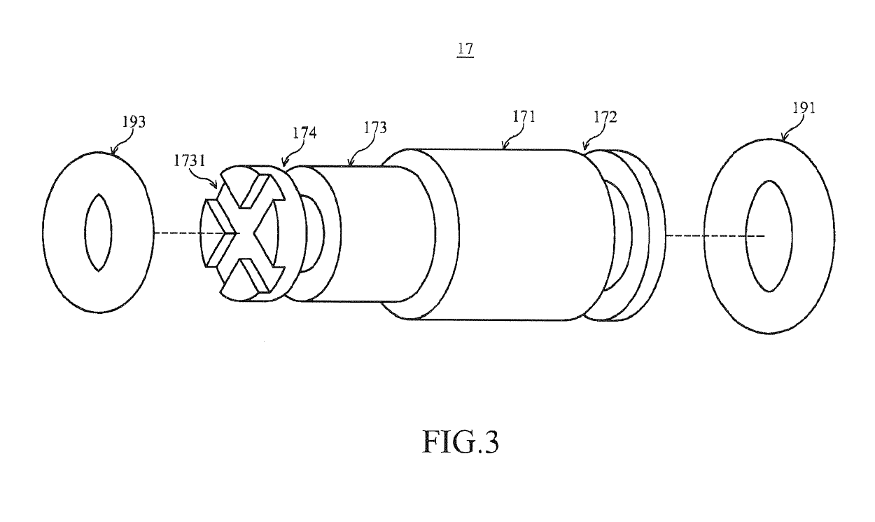

FIG. 4 is a cross-sectional diagram of the needleless positive-pressure connector in the closed state of the present invention; and

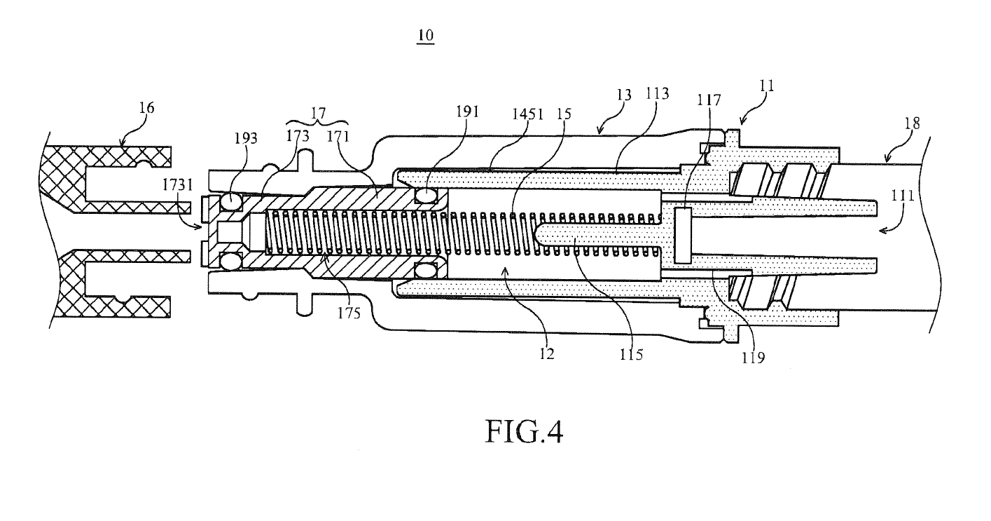

FIG. 5 is a cross-sectional diagram of the needleless positive-pressure connector in the open state of the present invention

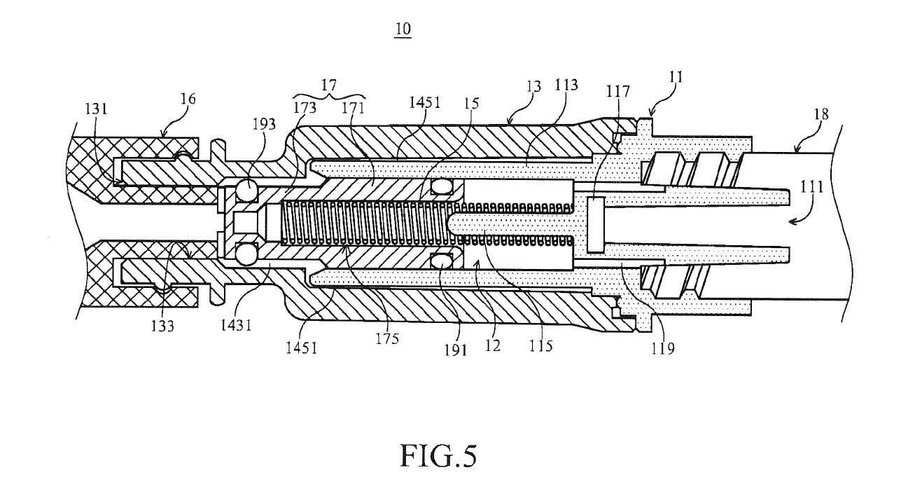

Although the embodiments of the present invention are illustrated in the figures by way of example, and described in detail in the text, various modifications and alternatives are still allowed. The figures of the present invention may be not drawn to scale. The figures and detailed description therefor may be only specific disclosure, and not used for limiting the present invention. In contrast, modifications, equivalent components, and substitutions on the basis of spirits and scopes of Claims are all covered by the present invention.

1. A needleless positive-pressure connector, comprising:
a connecting pipe comprising an outlet and an extension portion, wherein said extension portion is provided therein with a first accommodating room;
a casing pipe comprising an inlet and a second accommodating room, wherein said extension portion of said connecting pipe is located within said second accommodating room when said casing pipe and said connecting pipe are coupled;
a piston connected to said connecting pipe, a part of said piston being located within said first accommodating room, wherein said piston is used for displacing within said first accommodating room and said second accommodating room, as well as opening or closing said inlet; and
an elastic unit, located between said connecting pipe and said piston.

2. The needleless positive-pressure connector according to claim 1, wherein said connecting pipe comprises a projection portion located within said first accommodating room, while said piston comprises a depressed portion, as well as one end of said elastic unit is located at said projection portion, while the other end of said elastic unit is located within said depressed portion.

3. The needleless positive-pressure connector according to claim 1, wherein said piston is used for separating said first accommodating room from said second accommodating room.

4. The needleless positive-pressure connector according to claim 1, wherein said piston is used for separating said first accommodating room from said outlet

5. The needleless positive-pressure connector according to claim 1, further comprising at least one connecting hole connecting said second accommodating room to said outlet

6. The needleless positive-pressure connector according to claim 1, further comprising a gap located between said extension portion of said connecting pipe and said casing pipe.

7. The needleless positive-pressure connector according to claim 1, further comprising a first sealing unit and a second sealing unit located at two ends of said piston, respectively, said first sealing unit being located within said first accommodating room of said connecting pipe, said second sealing unit facing toward said inlet

8. The needleless positive-pressure connector according to claim 7, wherein said second sealing unit is adhered to an inner wall of said casing pipe, and said second accommodating room is separated from the outside to close said inlet.

9. The needleless positive-pressure connector according to claim 1, wherein said connecting pipe further comprises at least one vent connected with said first accommodating room.

10. The needleless positive-pressure connector according to claim 1, further comprising at least one notch located at a front end of said piston.
